(19) United States
(12) Patent Application Publication
(10) Pub. No.: US 2025/0044223 A1
(43) Pub. Date: Feb. 6, 2025

(54) CAPPED SEMICONDUCTOR BASED SENSOR AND METHOD FOR ITS FABRICATION

(71) Applicant: Senseair AB, Delsbo (SE)

(72) Inventor: Stephan Schröder, Bålsta (SE)

(21) Appl. No.: 18/719,554

(22) PCT Filed: Dec. 19, 2022
(86) PCT No.: PCT/SE2022/051206

§ 371 Date: Jun. 13, 2024

(30) Foreign Application Priority Data
Dec. 22, 2021 (SE) 2151590-3

(51) Int. Cl. G01N 21/3504; G01N 33/00; H01L 31/173; H01L 31/18

(52) U.S. Cl. CPC G01N 21/3504; G01N 33/0027; H01L 31/1876; H01L 31/173

(57) ABSTRACT

A method for fabricating semiconductor-based sensor devices and such a sensor device are described. The sensor devices comprise sensors comprising micro- and/or nanostructures which are in communication with the environment surrounding the sensor devices. The method comprises the steps of providing a semiconductor-based device wafer, fabricating a plurality of sensors on the semiconductor-based device wafer (1), providing (102) a capping wafer, attaching a first side of the capping wafer on the device wafer with each sensor arranged below a recess. The capping wafer comprises, between the recesses, a plurality of holes extending from the second side, wherein the holes are in fluid communication with the cavities by passages arranged between contact areas when the capping wafer has been attached to the device wafer. The method comprises the steps of injecting a liquid into the passages and the holes, forming, from the liquid, a gas permeable segment in the passages, and dividing the device wafer and the attached capping wafer into individual devices along lines through the holes.

### TECHNICAL FIELD

The present invention relates to a semiconductor-based sensor device and a method for its fabrication.

### BACKGROUND ART

Semiconductor-based sensor devices are increasingly used to sense various entities. By using semiconductor-based sensor devices the dimensions of the sensor devices may be kept small at the same time as the cost can be kept low. One type of semiconductor-based sensor devices are photonic gas sensors. Photonic gas sensors rely on the interaction of light with a gas and the measurement of the attenuation of the light by the interaction of light with the gas. One type of photonic sensor may comprise a photonic structure such as a waveguide, a light source and a detector. This type of sensor utilises the principle of exciting, with the light source, an electromagnetic field in the waveguide, which electromagnetic field comprises an evanescent wave. Typically, the evanescent wave refers to the portion of the electromagnetic field that propagates outside of the waveguide but is related to the propagating electromagnetic wave in the waveguide. The evanescent wave interacts with the gas surrounding the waveguide. The wavelength of the radiation is chosen to correspond to an absorption peak of the gas to be sensed, which is usually IR radiation. For most gases of interest, the absorption peak is in the infrared wavelength region. In order to minimize the effect, on the evanescent wave, of compromising factors other than the gas, the waveguide may be configured to be partially freehanging. This may be achieved by having the waveguide supported on posts/pillars extending from the surface of the substrate. Such a sensor consists of fragile micro- and/or nanostructures, in the form of, e.g., gratings for coupling of radiation into the waveguide, one or more waveguides, and posts/pillars. The sensor may also comprise one or more multimode interference (MMI) couplers/splitters and combiners, photonic resonators, such as one or more ring resonators or other photonic components. Such sensors can be fabricated in a semiconductor material using conventional semiconductor fabrication approaches, wherein a plurality of sensor devices are fabricated on a common wafer. This is a cost-effective fabrication method that has been broadly used for the fabrication of micro- and nanoelectronics. The handling of these devices during and after the fabrication is problematic and a common approach to protect the devices is wafer level packaging (WLP) that is primarily based on attaching a capping wafer on the device wafer, which contains the photonic gas sensor devices. By providing a capping wafer, the photonic structures of the sensors become protected from damages from the outside. In order to allow gas to come in contact with the waveguide it is necessary to provide gas entrances through the capping wafer or the device wafer.

After having attached the capping wafer, the device wafer and the attached capping wafer have to be divided into individual sensor devices. Such individualization is traditionally called singulation. Traditionally, the dividing process is performed with a rotating blade. During the dividing process with a rotating blade, particles and heat is generated. The process of sawing a wafer into a single chip is usually called die sawing or dicing. For cooling purposes, water is sprayed onto the blade and wafer during die sawing or dicing, i.e., the dividing process. To prevent water and/or particles from coming into contact with the waveguide the sensor devices need to be fully protected during the dividing process, such that water and particles cannot access the devices.

The problem of avoiding particles to enter a device exists also for micro-electromechanical systems, MEMS, which needs to be in contact with the atmosphere. Semiconductor based MEMS devices and photonic gas sensor devices, possibly with a partly freehanging waveguide, both constitute devices with micro and/or nano elements. MEMS devices are described in US 2006/0001114 A1, in which a lid is attached to a substrate with MEMS. Openings are provided in the lid, which openings are filled with a protective gel. After having attached the lid on the substrate with the MEMS a dividing process is performed for producing single chips. After the dividing process, the protective gel is removed such that the MEMS get in contact with the atmosphere.

When semiconductor-based sensor devices comprising micro- and/or nanostructures are used in a dusty environment, the dust affects the reliability and operation of the sensor devices negatively.

WO2016060619 A1 describes an optical waveguide structure and an optical gas sensor and methods of fabrication thereof.

US2020/0255285 A1 describes a micromechanical sensor device and corresponding production method.

### SUMMARY OF THE INVENTION

An object of the present invention is to provide a sensor device comprising micro- and/or nanostructures that rely on a gas exchange with the environment and a method for fabricating such a sensor device, wherein the sensor device is less sensitive to dust during operation than conventional sensor devices.

Another object of the present invention is to provide a sensor device comprising micro- and/or nanostructures that rely on a gas exchange with the environment and a method for fabricating such a sensor device, which is an alternative to the sensor devices according to the prior art.

At least one of these objects is fulfilled with a sensor device and a method according to the independent claims.

Further advantages are achieved with the features of the dependent claims.

According to a first aspect, a method is provided for fabricating semiconductor-based sensor devices with sensor parts comprising micro- and/or nanostructures, which are configured to interact with the environment surrounding the sensor devices. The method comprises the steps of providing a semiconductor-based device wafer, fabricating a plurality of sensor parts comprising micro- and/or nanostructures on different device areas on a device side of the device wafer, and providing a capping wafer comprising a first side and a second side and a plurality of recesses on the first side. The method also comprises the step of attaching the first side of the capping wafer on the device wafer with each sensor part arranged below a recess such that a cavity is formed between each recess and the device wafer. The method is characterized in that the capping wafer comprises, between the recesses, a plurality of holes extending from the second side, wherein the capping wafer is in contact with the device wafer in contact areas arranged at the periphery of the recesses, and wherein the holes are in fluid communication with the cavities by passages arranged between contact areas when the capping wafer has been attached to the device wafer. The method is characterized in that is further comprises the steps of injecting a liquid into the passages and the holes, forming, from the liquid, gas permeable segments in the passages, and dividing the device wafer and the attached capping wafer into individual devices along lines extending through the holes.

With a method according to the first aspect, the sensor part comprising micro- and/or nanostructures is protected from dust and particles originating from the dividing process. Dividing of the device wafer and the attached capping wafer into individual devices is normally performed using a saw blade, while simultaneously providing cooling water to the saw blade. During the dividing process a lot of particles are formed which are prevented from entering the cavity by the treated injected liquid. The gas permeable segment also protects the sensor part from contamination of dust during operation. The gas permeable segment also protects the sensor part from water during operation.

The passages may be formed by indentations in the capping wafer, wherein the indentations may be arranged between the recess and the holes when the capping wafer has been attached to the device wafer. Alternatively or additionally, the passages may be formed by indentations in the semiconductor-based device wafer.

At the positions of the passages, the gas permeable segments extend, in the direction perpendicular to the device wafer, from the device wafer to the capping wafer.

In the passages, the device wafer is free from contact with the capping wafer, i.e., the device wafer is not in contact with the capping wafer at the positions of the passages.

The sensor parts do not have to comprise all parts necessary for a functioning sensor but may comprise only the parts, which are configured to interact with the environment or any other combination of parts, i.e., waveguide and detector but no light source or waveguide and light source but no detector. An external light source or external detector might be coupled to the sensor device in different ways. One possibility is to configure a grating on the outside of the sensor device for coupling of light into the waveguide of the sensor device and/or for coupling of light out from the waveguide of the sensor device. The light source and/or the detector may be arranged in close proximity to the grating(s) on the outside of the sensor device. It is also possible to arrange a waveguide and/or an optical fiber between the light source and/or the detector and the grating(s) to allow the light source and/or detector to be placed further away from the sensor device.

The sensor parts and the corresponding recess of the capping wafer may be configured such that the sensor parts are free from contact with the capping wafer. This allows a wide variety of sensor parts to be used.

Due to the gas permeable segments in the passages, gas may enter the cavity in which it is interacting with the sensor part comprising the micro- and/or nanostructures.

The feature that the holes are in fluid communication with the cavities by passages means that the passages are connected to the holes as well as to the cavities. Liquid that is injected through the holes flows to the passages.

The capping wafer may be made of many different materials, such as, e.g., a semiconductor material, a ceramic material or a polymer.

The step of forming, from the liquid, a gas permeable segments in the passages, may comprise curing the liquid by heat treatment to solidify the liquid. Alternatively, the liquid may be such that it solidifies after a certain time, either be evaporation of a solvent or by reaction between two components in the liquid.

The liquid may be a polymer, wherein the cured polymer is gas permeable and allows diffusion of gas or is a porous polymer that allows transmission of gas.

The liquid may be a matrix of a polymer and particles, wherein the forming, from the liquid, a gas permeable segment in the passages, comprises the steps of curing the liquid by heat treatment to solidify the liquid, and completely or partially removing the polymer such that the particles remain as the gas permeable segment. Preferably, the step of dissolving the polymer is performed after the step of dividing the wafer and the capping wafer into individual sensor devices. The particles may be made of different materials such as, e.g., a second polymer or ceramics. The particles may be porous or non-porous. Ceramic particles are favourable in that the gas permeable segment becomes heat resistant.

The sensor parts comprising micro- and/or nanostructures may be a photonic gas sensor parts comprising photonic structures such as, e.g., a waveguide, which may be at least partly free hanging. This is a type of sensor part which requires contact with gas for the sensing and which is sensitive for contamination with particles and dust from the dividing process. If particles and dust are allowed to come in contact with the waveguide, the particles, the dust and water, will affect the device operation and reliability negatively and eventually lead to a malfunction of the device.

The sensor part comprising micro- and/or nanostructures may be a photonic gas sensor parts and may comprise a light source, a detector, and waveguides, which may be at least partially free hanging. This is a type of sensor part which requires contact with gas for the sensing and which is sensitive for contamination with particles, dust and water from the dividing. If particles, dust or water is allowed to come in contact with the waveguide, the particles and the dust will contribute to a malfunction of the sensor. The light source may be an infrared, IR, light source. The light source may be an IR light emitting diode, a laser, or a resistive emitter, which is a metal or another conductive material with heats up when a current is forced through it.

The waveguide may be suspended on pillars/posts extending from the device wafer, wherein the waveguide is free hanging between the pillars/posts. By having the waveguide free hanging between the pillars/posts the attenuation of the radiation due to surrounding material is minimized. Such a sensor part is strongly affected by dust and particles coming into contact with the waveguide.

The sensor device is not limited to being a gas sensor comprising a photonic structure, but may be any type of sensor that needs to be in contact with the surrounding environment. Another example on a sensor, which needs to be in contact with the environment, is a pressure sensor or a humidity sensor. A pressure sensor or a humidity sensor may also be based on micro- and/or nanostructures.

According to a second aspect a semiconductor-based sensor device is provided, which comprises a micro- and/or nanostructure, which is configured to interact with the environment surrounding the sensor device. The sensor device comprises a semiconductor-based substrate, a sensor part comprising micro- and/or nanostructures arranged on a device side of the substrate, and a cap comprising a recess, wherein the cap is arranged on the device side of the substrate with the sensor part arranged below the recess such that a cavity is formed between the recess and the substrate. The sensor device is characterized in that the cap is in contact with the substrate in contact areas arranged at the periphery of the recess, and in that the sensor device also comprises at least one gas permeable segment arranged between the substrate and the cap and between the contact areas, to provide a gas passage between the cavity and the environment surrounding the sensor device.

As was mentioned for the first aspect the sensor comprising micro- and/or nanostructures is protected from dust and particles by the gas permeable segment.

The gas permeable segment also protects the sensor from water during operation.

Said at least one gas permeable segment may be arranged in a passage in the form of an indentation in the cap, wherein the indentation may be arranged between the recess and the environment surrounding the semiconductor-based sensor device. Alternatively or additionally, the passages may be formed by indentations in the semiconductor-based device wafer.

The at least one gas permeable segment extends, in the direction perpendicular to the semiconductor-based substrate, from the semiconductor-based substrate to the cap.

At the at least one gas permeable segment the semiconductor-based substrate is free from contact with the cap, i.e., the semiconductor-based substrate is not in contact with the cap at the positions of the at least one gas permeable segment.

The sensor parts and the recess of the cap may be configured such that the sensor parts are free from contact with the cap. This allows a wide variety of sensor parts to be used.

The sensor parts do not have to comprise all parts necessary for a functioning sensor but may comprise only the parts which are configured to interact with the environment.

The feature that the holes are in fluid communication with the cavities by passages means that the passages are connected to the holes as well as to the cavities. Liquid that is injected through the holes flows to the passages. The capping wafer may be made of many different materials, such as, e.g., a semiconductor material, a ceramic material or a polymer.

The sensor part comprising micro- and/or nanostructures may be a photonic gas sensor comprising photonic structures such as a waveguide, which may be at least partly free hanging. This is a type of sensor which requires contact with gas for the sensing and which is sensitive for contamination with particles and dust during operation. If particles, dust, and water are allowed to come in contact with the photonic structures the particles, dust and water will contribute to will negatively affect the operation and reliability of the sensor device.

The waveguide may be suspended on pillars/posts extending from the device wafer, wherein the waveguide is free hanging between the pillars/posts. By having the waveguide suspended in this way on the pillars/posts, the attenuation of the radiation due to surrounding material is minimized. Such a waveguide is strongly affected by dust, particles and water coming into contact with the waveguide.

The sensor part comprising micro- and/or nanostructures may be a photonic gas sensor part and may comprise a light source, a detector, and a waveguide, which may be at least partially free hanging. This is a type of sensor which requires contact with gas for the sensing and which is sensitive for contamination with particles, dust and water from the dividing. If particles, dust or water is allowed to come in contact with the waveguide, the particles and the dust will contribute to a malfunction of the sensor. The light source may be an infrared, IR, light source. The light source may be an IR light emitting diode, a laser, or a resistive emitter, which is a metal or another conductive material with heats up when a current is forced through it.

The sensor device is not limited to being a gas sensor comprising photonic structures, but may be any type of sensor that needs to be in contact with the surrounding environment. Another example on a sensor, which needs to interact with the environment, is a pressure sensor or a humidity sensor. A pressure sensor or humidity may also be based on micro- and/or nanostructures.

The gas permeable segment may be a gas permeable polymer, which allows diffusion of gas or may be a porous polymer that allows transmission of gas. According to another embodiment, the gas permeable segment comprises particles. The latter alternative may provide a better exchange of gas. The particles may be made of different materials such as, e.g., a second polymer or ceramics. Ceramic particles are favourable in that the gas permeable segment becomes heat resistant.

### BRIEF DESCRIPTION OF THE DRAWINGS

FIG. 1a-f illustrates the method for production of a semiconductor-based sensor device.

FIG. 2 shows in a perspective view a semiconductor-based device wafer with a capping wafer attached, before injecting a liquid into the holes in the capping wafer.

FIG. 3 shows in a perspective view a semiconductor-based device wafer with a capping wafer attached, after injecting a liquid into the holes in the capping wafer.

FIG. 4 shows in a perspective view a sensor device after dividing of the semiconductor-based device wafer with the attached capping wafer of FIG. 3.

FIG. 5 is a flow diagram of a method according to the present invention.

### DETAILED DESCRIPTION

In the following detailed description of embodiments, similar features in the different drawings are denoted with the same reference numerals. The drawings are not drawn to scale.

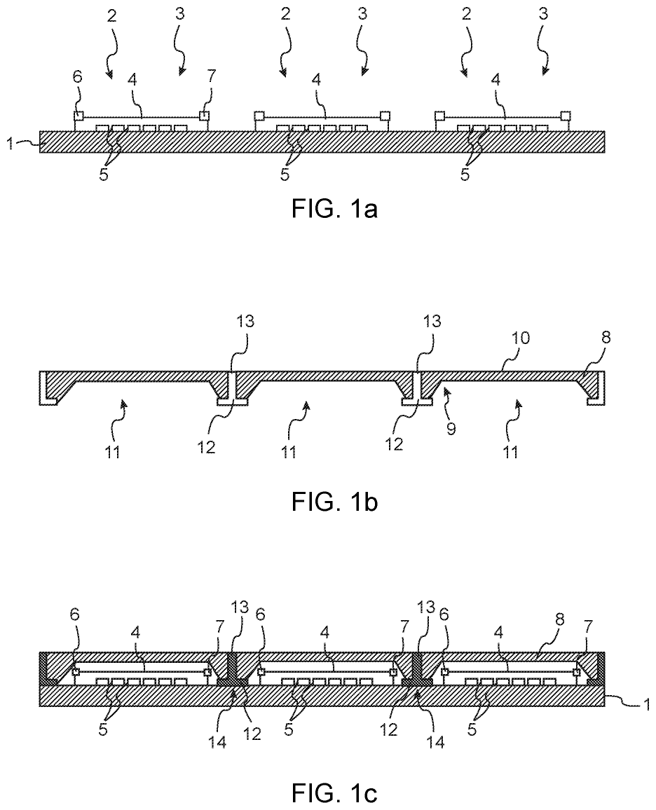

FIG. 1a-f illustrates the method for production of a semiconductor-based sensor device. FIG. 1a shows in cross section a semiconductor-based device wafer 1. A plurality of sensors 2 comprising micro- and/or nanostructures have been fabricated on different device areas 3 on the semiconductor-based wafer 1. The semiconductor may be silicon, silicon nitride, silicon carbide, gallium arsenide or any other semiconductor on which a sensor may be fabricated. In the embodiment of FIG. 1a, the sensors are infrared gas sensors. The sensor parts of each sensor comprise a photonic structure in the form of a waveguide 4, which is suspended on pillars/posts 5 extending from the device wafer 1 to the waveguide 4, such that the waveguide is free hanging between the pillars/posts is at least partly free hanging. In the embodiment of FIG. 1, the sensor parts of each sensor 2 also comprises a light source 6 in one end of the waveguide 4 and a photodetector 7 in the other end of the waveguide 4. The light source 6 is configured to inject radiation into the waveguide 4 towards the photodetector 7. The wavelength of the light from the light source is chosen to correspond to the absorption peaks of the gases to be detected by the sensor. The wavelength is usually IR radiation. The light source 6 may be, e.g., a light emitting diode, LED, a laser diode, or a resistive element. The cross sectional dimensions of the waveguide 4 are such that the radiation in the waveguide 4 forms an evanescent wave, which interacts with the gas surrounding the waveguide. Thus, the attenuation of the radiation in the waveguide 4 will depend on the concentration of the gas surrounding the waveguide.

It is possible to have the light source 6 and/or the photodetector 7 arranged outside the sensor device.

According to alternative embodiments, the sensor is not a gas sensor as shown in FIG. 1a. FIG. 1b shows a capping wafer 8 comprising a first side 9 and a second side 10 and a plurality of recesses 11 on the first side 9. The capping wafer 8 comprises passages 12 at the second side 10, which extends between recesses 11, and holes 13, which extends between the first side 9 and the passages 12 at the second side 10. The recesses, the passages and the holes may be fabricated using standard etching or ablation techniques. The passages 12 are formed by indentations in the capping wafer 8, wherein the indentations are arranged between the recesses 11 and the holes 13. Alternatively, the passages may be formed by indentations 12′ in the semiconductor-based device wafer 1 as is shown by the dotted lines in FIGS. 1e and 1f.

At the positions of the passages 12, the gas permeable segments extend, in the direction perpendicular to the device wafer, from the device wafer 1 to the capping wafer 8.

In the passages 12 the device wafer 1 is free from contact with the capping wafer 8, i.e., the device wafer 1 is not in contact with the capping wafer 8 at the positions of the passages 12.

In FIG. 1c, the first side of the capping wafer 8 has been attached on the device wafer 1 with each sensor 2 arranged below a recess 11 such that a cavity is formed between each recess 11 and the device wafer 1. In Figure the plurality of holes 13 and the passages 12 have been filled with a liquid polymer. The liquid polymer is heat treated to cure the polymer to solidify the polymer. Alternatively, the liquid polymer may be a polymer that solidifies after a certain time, either by evaporation of a solvent or by reaction between two components.

As can be seen in FIG. 1c the capping wafer 8 is in contact with the device wafer 1 in contact areas 14 arranged at the periphery of the recesses 11. The holes 13 are in fluid communication with the cavities by the passages 12 arranged between contact areas when the capping wafer has been attached to the device wafer 1. The sensor parts 4 and the corresponding recess 11 of the capping wafer 8 may be configured such that the sensor parts 4 are free from contact with the capping wafer 8. This allows a wide variety of sensor parts to be used, such as the above-described sensor comprising a photonic structure in the form of a waveguide 4, which is suspended on pillars/posts 5 extending from the device wafer 1 to the waveguide 4, such that the waveguide is free hanging between the pillars/posts is at least partly free hanging. Such sensors are sensitive to contact with surrounding material.

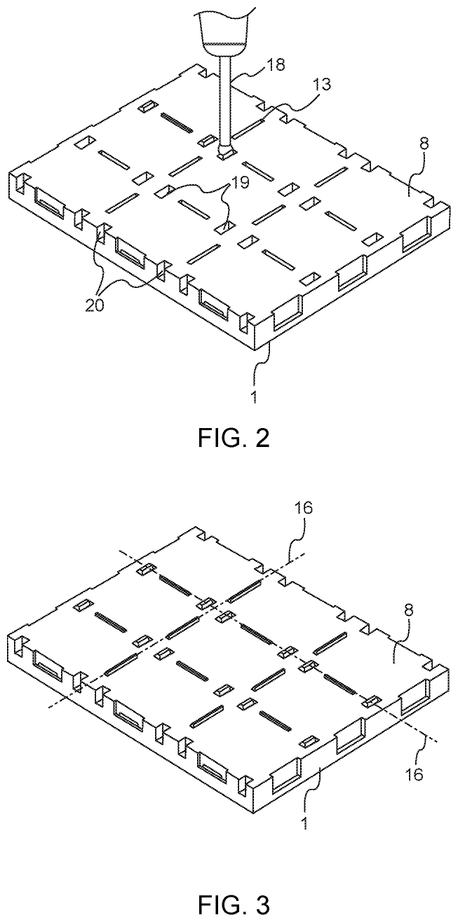

In FIG. 1d it is illustrated how the device wafer 1 and the attached capping wafer 8 is divided into individual devices 15 along lines 16 (FIG. 3) through the holes 13. The dividing may be performed using a rotating saw blade 17. Alternatively, the dividing may be performed by laser ablation techniques. During dividing, particles are formed. The solidified polymer in the passages 12 prevents the particles, formed by the rotating saw blade, from passing the passages 12 into the cavities formed by the recesses 11.

The liquid polymer is treated to form gas permeable segments in the passages 12.

The gas permeable segments in the passages may be formed in many different ways.

According to one embodiment, the polymer is chosen such that it after curing forms a gas permeable solid polymer. Thus, the gas permeable segments are formed after curing.

According to one embodiment, the gas permeable polymer is configured to allow diffusion of gas.

According to another embodiment, the gas permeable polymer is a porous polymer that allows transmission of gas.

According to another embodiment, the liquid polymer contains particles. After injection, the liquid polymer with the particles is heat treated to cure the polymer to solidify the liquid polymer. After curing, the cured polymer is partially or completely removed by dissolution, leaving the particles as a porous segment. Alternatively, the polymer may solidify after a certain time without heating. The particles may be of many different materials, such as, e.g., a second polymer or ceramic. In case the particles are ceramic, the resulting gas permeable segment becomes heat resistant.

FIG. 1e shows a sensor device 15 according to an embodiment, after dividing the device wafer 1 and the capping wafer 8 into individual sensor devices 15. The gas permeable segments 21 are shown as a gas permeable polymer in FIG. 1e as is more clearly illustrated in the enlargement in FIG. 1e.

FIG. 1f shows a sensor device 15 according to another embodiment, after dividing the device wafer 1 and the capping wafer 8 into individual sensor devices 15 such that the individual sensor device 15 comprises a substrate 1′ and a cap 8′. A photonic structure 4 in the form of a waveguide is arranged in the cavity formed by the recess 11 of the cap 8′ and the substrate 1′. The gas permeable segments 21 are shown as ceramic particles forming a porous segment 21 in FIG. 1f as is more clearly illustrated in the enlargement in FIG. 1e.

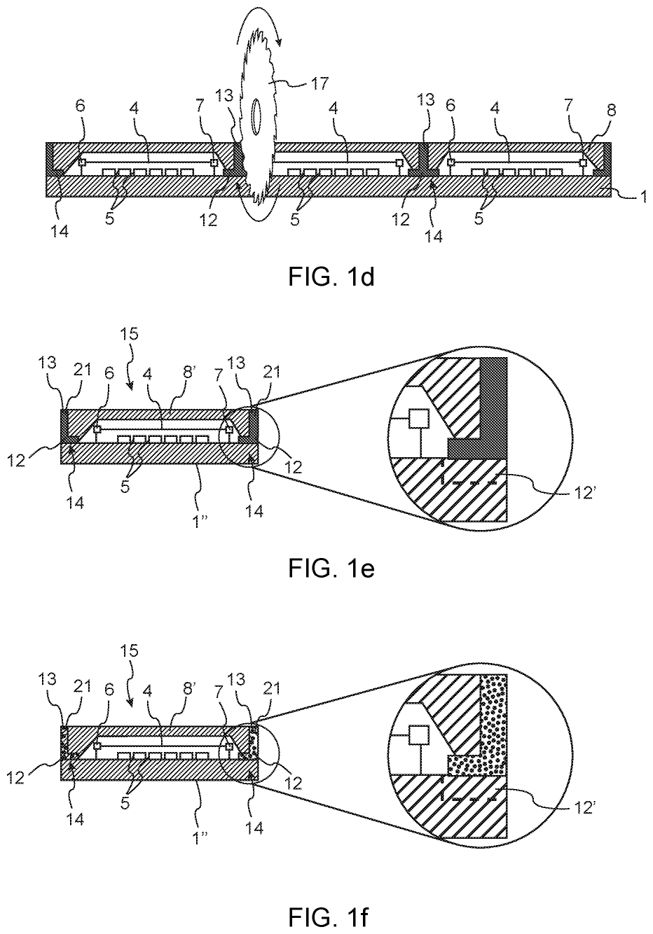

FIG. 2 illustrates the device wafer 1 on which the capping wafer 8 has been attached, during the step of injection of a liquid polymer in the holes 13 of the capping wafer 8. The liquid polymer is dispensed from a nozzle 18. Also seen in FIG. 2 are contact holes 19, which define contact areas 20 on the device wafer 1. Electrical wires are to be connected to the contact areas 20 on the device wafer 1.

FIG. 3 illustrates the device wafer 1 on which the capping wafer 8 has been attached, after the step of injection of a matrix of a liquid polymer and particles in the holes 13 and the contact holes of the capping wafer 8. The device wafer 1 and the capping wafer 8 are divided into individual sensor devices 15 along the lines 16.

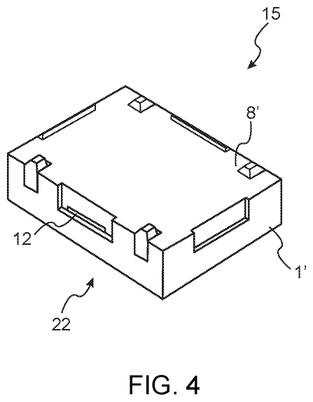

FIG. 4 illustrates a sensor device 15 after dividing into individual sensor devices 15. In case the polymer is to be removed the step of removing the polymer is performed after the step of dividing the device wafer 15 and the attached capping wafer 8 into individual devices 15 comprising a semiconductor substrate 1′ and a cap 8′. In FIG. 4, the injected liquid has been removed from the front side 22 to show the passage 12.

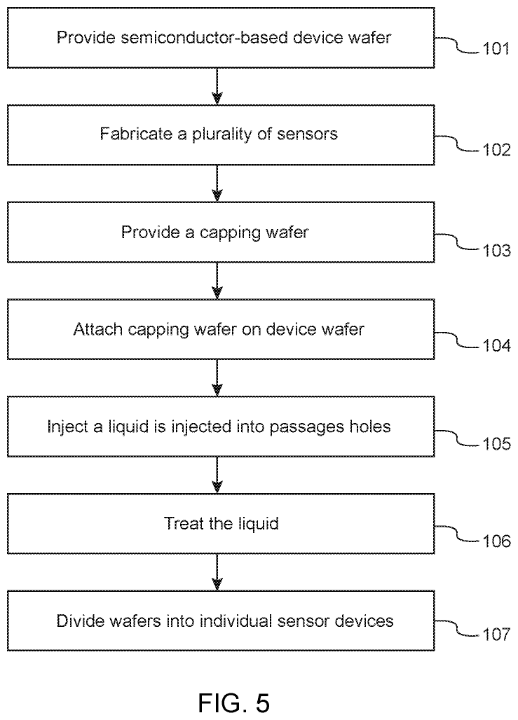

FIG. 5 is a flow diagram method for fabricating semiconductor-based sensor devices comprising micro- and/or nanostructures, which are in communication with the environment surrounding the sensor devices of the method according to an embodiment of the invention.

The flow diagram will be described with reference to FIGS. 1-4 described above. In a first step 101, a semiconductor-based device wafer 1 is provided. In a second step 102 a plurality of sensors 2 comprising micro- and/or nanostructures are fabricated on different device areas 3 on the semiconductor-based device wafer 1. In a third step 103 a capping wafer 8 comprising a first side 9 and a second side 10 and a plurality of recesses on the first side 9, is provided. In a fourth step 104 the first side of the capping wafer 11 is attached on the device wafer 1 with each sensor 2 arranged below a recess 11 such that a cavity is formed between each recess 11 and the device wafer 1. In a fifth step 105 a liquid is injected into the passages 12 and the holes 13. In a sixth step 106, the liquid is formed into a gas permeable segment in the passages 12. Depending on the liquid, the forming is different. According to one alternative, the liquid comprises a polymer. The polymer may be solidified either by heating or by waiting. In a seventh step 107, the device wafer 1 and the attached capping wafer 8 are divided into individual devices 15 along lines 16 through the holes 13.

Depending on how the gas permeable segment is formed, the method may comprise additional steps. According to one embodiment, the liquid is a matrix of a polymer and particles. The particles may be of many different materials. The particles may be, e.g., ceramic particles, or polymer particles. After the step of dividing 107, the device wafer 1 and the attached capping wafer 8 are divided into individual devices 15. The method according to this embodiment also comprises the step of removing 108 the polymer such that only the particles remain in the passages. In case the remaining particles are ceramic, the gas permeable segment is heat resistant. Ceramic particles may be of, e.g., alumina ceramics, silicon carbide ceramics, or zirconia oxide ceramics

If the gas permeable segments in the passages 12 are made of a polymer through which gas may diffuse the step of removing 108 the polymer is not performed. Examples on polymers, which allow diffusion of gas through it are, e.g., polydimethylsiloxane, PDMS, which is well known to have a rather high diffusion coefficient of CO2, and polymethyl methacrylate, PMMA.

The described embodiments may be amended in many ways without departing from the scope of the invention, which is limited only by the appended claims.

1. A method for fabricating semiconductor-based sensor devices with sensors comprising micro- and/or nanostructures which are configured to interact with the environment surrounding the sensor devices, comprising the steps of
providing a semiconductor-based device wafer,
fabricating a plurality of sensor parts comprising micro- and/or nanostructures on different device areas on a device side of the device wafer,
providing a capping wafer comprising a first side and a second side and a plurality of recesses on the first side,
attaching the first side of the capping wafer on the device wafer with each sensor part arranged below a recess such that a cavity is formed between each recess and the device wafer,
characterized in that the capping wafer comprises, between the recesses, a plurality of holes extending from the second side, wherein the capping wafer is in contact with the device wafer in contact areas arranged at the periphery of the recesses, and wherein the holes are in fluid communication with the cavities by passages arranged between contact areas when the capping wafer has been attached to the device wafer, wherein the method further comprises the steps of
injecting a liquid into the passages and the holes,
forming, from the liquid, a gas permeable segment in the passages, and
dividing the device wafer and the attached capping wafer into individual sensor devices along lines extending through the holes.

2. The method according to claim 1, wherein the step of forming the liquid into a gas permeable segment comprises curing the liquid by heat treatment to solidify the liquid.

3. The method according to claim 2, wherein the liquid is a polymer and wherein the cured polymer is gas permeable and allows diffusion of gas or is a porous polymer that allows transmission of gas.

4. The method according to claim 2, wherein the liquid is a matrix of a polymer and ceramic particles, wherein the forming, from the liquid, a gas permeable segment in the passages, comprises the steps
curing the liquid by heat treatment to solidify the liquid, and
at least partially removing the polymer such that the particles remain as the gas permeable segment.

5. The method according to claim 4, wherein the step of removing the polymer is performed after the step of dividing the device wafer and the attached capping wafer into individual sensor devices.

6. The method according to claim 5, wherein the sensor parts comprising micro- and/or nanostructures are photonic gas sensor parts comprising photonic structures.

7. The method according to claim 6, wherein the photonic structure comprises a waveguide, which is suspended on pillars/posts extending from the device wafer, wherein the waveguide is free hanging between the pillars/posts.

8. A semiconductor-based sensor device comprising a micro- and/or nanostructure which is configured to interact with the environment surrounding the sensor device, comprising
a semiconductor-based substrate,
a sensor part comprising a micro- and/or nanostructure arranged on a device side of the substrate,
a cap comprising a recess, wherein the cap is arranged on the device side of the substrate with the sensor part arranged below the recess such that a cavity is formed between the recess and the substrate,
characterized in that the cap is in contact with the substrate in contact areas arranged at the periphery of the recess, and in that the sensor device also comprises at least one gas permeable segment arranged between the substrate and the cap and between the contact areas, to provide a gas passage between the cavity and the environment surrounding the sensor device.

9. The sensor device according to claim 8, wherein the micro- and/or nanostructure is a photonic gas sensor part comprising a photonic structure.

10. The sensor device according to claim 9, wherein the photonic structure comprises a waveguide, which is suspended on pillars/posts extending from the substrate, wherein the waveguide is free hanging between the pillars/posts.

11. The sensor device according to claim 8, wherein the gas permeable segment is a gas permeable polymer and allows diffusion of gas or is a porous polymer that allows transmission of gas.

12. The sensor device according to claim 10, wherein the gas permeable segment comprises particles.
